(19) United States
(12) Patent Application Publication
(10) Pub. No.: US 2009/0075312 A1
(43) Pub. Date: Mar. 19, 2009

(54) ASSESSING COLORECTAL CANCER BY MEASURING OSTEOPONTIN AND CARCINOEMBRYONIC ANTIGEN

(76) Inventors: Norbert Wild, Geretsried (DE); Veit Peter Grunert, Muenchen (DE); Johann Karl, Peissenberg (DE); Jarema Peter Kochan, Towaco, NJ (US); Peter Stegmueller, Am Bahnhoffeld (DE); Michael Tacke, Muenchen (DE)

Correspondence Address: ROCHE DIAGNOSTICS OPERATIONS INC., 9115 Hague Road, Indianapolis, IN, 46250-0457, US

(21) Appl. No.: 12/141,278

(22) Filed: Jun. 18, 2008

Related U.S. Application Data
(63) Continuation of application No. PCT/EP2006/012218, filed on Dec. 19, 2006.

(30) Foreign Application Priority Data
Dec. 22, 2005 (EP) 05028126.0

(51) Int. Cl. C12Q 1/48; G01N 33/00

(52) U.S. Cl. 435/15; 436/86

(57) ABSTRACT

The present invention relates to a method aiding in the assessment of colorectal cancer (=CRC). It discloses the use of a marker combination comprising osteopontin and carcinoembryonic antigen in the assessment of colorectal cancer. Furthermore, it especially relates to a method for assessing colorectal cancer from a liquid sample, derived from an individual by measuring at least the markers osteopontin and carcinoembryonic antigen in said sample. The marker combination comprising osteopontin and carcinoembryonic antigen can, e.g., be used in the early detection of colorectal cancer or in the surveillance of patients who undergo therapy, e.g., surgery.

### RELATED APPLICATIONS

This application is a continuation of PCT/EP2006/012218 filed Dec. 19, 2006 and claims priority to EP 05028126.0 filed Dec. 22, 2005.

### FIELD OF THE INVENTION

The present invention relates to a method aiding in the assessment of colorectal cancer (=CRC). It discloses the use of a marker combination comprising osteopontin and carcinoembryonic antigen in the assessment of colorectal cancer. Furthermore, it especially relates to a method for assessing colorectal cancer from a liquid sample, derived from an individual by measuring at least the markers osteopontin and carcinoembryonic antigen in said sample. The marker combination comprising osteopontin and carcinoembryonic antigen can, e.g., be used in the early detection of colorectal cancer or in the surveillance of patients who undergo therapy, e.g., surgery.

### BACKGROUND

Cancer remains a major public health challenge despite progress in detection and therapy. Amongst the various types of cancer, colorectal cancer (=CRC) is one of the most frequent cancers in the Western world.

Colorectal cancer most frequently progresses from adenomas (polyps) to malignant carcinomas. The different stages of CRC used to be classified according to Dukes' stages A to D.

The staging of cancer is the classification of the disease in terms of extent, progression, and severity. It groups cancer patients so that generalizations can be made about prognosis and the choice of therapy.

Today, the TNM system is the most widely used classification of the anatomical extent of cancer. It represents an internationally accepted, uniform staging system. There are three basic variables: T (the extent of the primary tumor), N (the status of regional lymph nodes) and M (the presence or absence of distant metastases). The TNM criteria are published by the UICC (International Union Against Cancer)(Sobin, L. H. and Fleming, I. D., Cancer 80 (1997) 1803-1804).

What is especially important is that early diagnosis of CRC translates to a much better prognosis. Most malignant tumors of the colorectum appear to arise from benign tumors, i.e. from adenoma. Therefore, best prognosis has those patients diagnosed at the adenoma stage. Patients diagnosed as early as in stage Tᵢₛ, N0, M0 or T1-3; N0; M0, if treated properly have a more than 90% chance of survival 5 years after diagnosis as compared to a 5-years survival rate of only 10% for patients diagnosed when distant metastases are already present.

In the sense of the present invention early diagnosis of CRC refers to a diagnosis at a pre-malignant state (adenoma) or at a tumor stage where no metastases at all (neither proximal nor distal), i.e., adenoma, Tᵢₛ, N0, M0 or T1-4; N0; M0are present. Tᵢₛ denotes carcinoma in situ.

It is further preferred, that CRC is diagnosed when it has not yet fully grown through the bowel wall and thus neither the visceral peritoneum is perforated nor other organs or structures are invaded, i.e., that diagnosis is made at stage Tᵢₛ, N0, M0 or T1-3; N0; M0 (=Tᵢₛ-3; N0; M0).

The earlier cancer can be detected/diagnosed, the belter is the overall survival rate. This is especially true for CRC. The prognosis in advanced stages of tumor is poor. More than one third of the patients will die from progressive disease within five years after diagnosis, corresponding to a survival rate of about 40% for five years. Current treatment is only curing a fraction of the patients and clearly has the best effect on those patients diagnosed man early stage of disease.

With regard to CRC as a public health problem, it is essential that more effective screening and preventative measures for colorectal cancer be developed.

The earliest detection procedures available at present for colorectal cancer involve using tests for fecal occult blood or endoscopic procedures. However, significant tumor size must typically exist before fecal blood is detected. The sensitivity of the guaiac-based fecal occult blood tests is ˜26%, which means 74% of patients with malignant lesions will remain undetected (Ahlquist, D.A., Gastroenterol. Clin. North Am. 26 (1997) 41-55). The visualization of precancerous and cancerous lesions represents the best approach to early detection, but colonoscopy is invasive, with significant costs, risks, and complications (Silvis, S. E., et al., JAMA 235 (1976) 928-930; Geenen, J. E., et al., Am. J. Dig. Dis. 20 (1975) 231-235; Anderson, W. F., et al., J. Natl. Cancer Institute 94 (2002) 1126-1133).

In order to be of clinical utility a new diagnostic marker as a single marker should be at least as good as the best single marker known in the art. Or, a new marker should lead to a progress in diagnostic sensitivity and/or specificity either if used alone or in combination with one or more other markers, respectively. The diagnostic sensitivity and/or specificity of a test is best assessed by its receiver-operating characteristics, which will be described in detail below.

The clinical utility of biochemical markers in colorectal cancer has recently been reviewed by the European Group on Tumor Markers (EGTM) (Duffy, M. J., et al., Eur. J. Cancer 39 (2003) 718-727).

At present, primarily diagnostic blood tests based on the detection of carcinoembryonic antigen (CEA), a tumor-associated glycoprotein, are available to assist diagnosis in the field of CRC. CEA is increased in 95% of tissue samples obtained from patients with colorectal, gastric, and pancreatic cancers and in the majority of breast, lung, and head and neck carcinomas (Goldenberg, D. M., et al., J. Natl. Cancer Inst. (Bethesda) 57 (1976) 11-22). Elevated CEA levels have also been reported in patients with nonmalignant disease, and many patients with newly defected colorectal cancer have normal CEA levels in the serum, especially during the early stage of the disease (Carriquiry, L. A., and Pineyro, A., Dis. Colon Rectum 42 (1999) 921-929; Herrera, M. A., et al., Ann. Surg. 183 (1976) 5-9; Wanebo, H. J., et al., N. Engl. J. Med. 299 (1978) 448-451; Wanebo, H. J., et al., supra). The utility of CEA as measured from serum or plasma in detecting recurrences is reportedly controversial and has yet to be widely applied (Martell, R. E., et al., Int. J. Biol. Markers 13 (1998) 145-149; Moertel, C. G., et al., JAMA 270 (1993) 943-947).

In light of the available data, serum CEA determination possesses neither the sensitivity nor the specificity to enable its use as a screening test for colorectal cancer in the asymptomatic population (Reynoso, G., et al., JAMA 220 (1972) 361-365; Sturgeon, C, Clinical Chemistry 48 (2002) 1151-1159).

Whole blood, serum or plasma are the most widely used sources of sample in clinical routine. The identification of an early CRC tumor marker that would aid in the reliable cancer detection or provide early prognostic information could lead to a diagnostic assay that would greatly aid in the diagnosis and in the management of this disease. Therefore, an urgent clinical need exists to improve the in vitro assessment of CRC. It is especially important to improve the early diagnosis of CRC, since for patients diagnosed early on chances of survival are much higher as compared to those diagnosed at a progressed stage of disease.

It was the task of the present invention to investigate whether a biochemical marker can be identified which may be used in assessing CRC.

Surprisingly, it has been found that use of a marker combination comprising osteopontin and carcinoembryonic antigen can at least partially overcome the problems known from the state of the art.

### SUMMARY OF THE INVENTION

The present invention relates to a method for assessing colorectal cancer in vitro comprising the steps of measuring in a sample the concentration of osteopontin, measuring in the sample the concentration carcinoembryonic antigen, and optionally measuring one or more other marker of colorectal cancer, and combining the concentration determined for osteopontin, carcinoembryonic antigen and optionally the one or more other marker of colorectal cancer, respectively, for assessing colorectal cancer.

Also disclosed is the use of the marker combination osteopontin and carcinoembryonic antigen in the assessment of colorectal cancer and the use of a marker panel comprising osteopontin, carcinoembryonic antigen, and one or more other marker for colorectal cancer in the assessment of colorectal cancer.

The invention further relates to a kit for performing the method of assessing CRC according to the present invention comprising the reagents required to specifically measure osteopontin and carcinoembryonic antigen.

### DETAILED DESCRIPTION OF THE INVENTION

In a preferred embodiment the present Invention relates to a method for assessing colorectal cancer in vitro comprising the steps of a) measuring in a sample the concentration of osteopontin, b) measuring in the sample the concentration carcinoembryonic antigen, and, c) optionally measuring of one or more other marker of colorectal cancer, and d) combining the concentration determined in steps (a), (b), and optionally the concentration(s) determined in step (c) for assessing colorectal cancer.

### Osteopontin (OPN)

OPN is found in normal plasma, urine, milk and bile (U.S. Pat. No. 6,414,219; U.S. Pat. No. 5,695,761; Denhardt, D. T. and Guo, X., FASEB J. 7 (1993) 1475-1482; Oldberg, A., et al., PNAS 83 (1986) 8819-8823; Oldberg, A., et al., J. Biol. Chem. 263 (1988) 19433-19436; Giachelli, CM., et al., Trends Cardiovasc. Med. 5 (1995) 88-95). The human OPN protein and cDNA have been isolated and sequenced (Kiefer M. C., et al., Nucl. Acids Res. 17 (1989) 3306).

OPN functions in cell adhesion, chemotaxis, macrophage-directed interleukin-10 (IL-10) suppression, stress-dependent angiogenesis, prevention of apoptosis, and anchorage-independent growth of tumor cells by regulating cell-matrix interactions and cellular signaling through binding with integrin and CD44 receptors. While constitutive expression of OPN exists in several cell types, induced expression has been detected in T-lymphocytes, epidermal cells, bone cells, macrophages, and tumor cells in remodeling processes such as inflammation, ischemia-reperfusion, bone resorption, and tumor progression (reviewed by Wai, P. Y. & Kuo P. C. J. Surg. Res. 121 (2004) 228-241).

OPN is known to interact with a number of integrin receptors. Increased OPN expression has been reported in a number of human cancers, and its cognate receptors (av-b3i av-b,5, and av-b1 integrins and CD44) have been identified. In vitro studies by Irby, R. B., et a)., Clin. Exp. Metastasis 21 (2004) 515-523 indicate that both endogenous OPN expression (via stable transfection) as well as exogenous OPN (added to culture medium) enhanced the motility and invasive capacity of human colon cancer cells in vitro. OPN appeared to regulate motility though interaction with CD44. OPN expression also reduced intercellular (homotypic) adhesion, which is regarded as a characteristic of metastatic cancer cells. Stable transfection of four poorly tumorigenic human colon cancer cell lines with OPN also resulted in enhanced tumorigenicity in vivo with increased proliferation and increased CD31 positive micro vessel counts, concordant with the degree of OPN expression.

Mor, G., et al., Proc. Natl. Acad. Sci. USA 102 (2005) 7677-7682 report a blood (serum) test for the early diagnosis of epithelial ovarian cancer based on the simultaneous quantization Of OPN and three other analytes.

In a preferred embodiment the present invention relates to a method for assessing CRC in vitro by biochemical markers, comprising measuring in a sample the concentration of osteopontin and using the concentration determined in the assessment of CRC.

### Carcinoembryonic Antigen (CEA)

CEA (carcinoembryonic antigen) is a monomeric glycoprotein (molecular weight approx. 180.000 Dalton) with a variable carbohydrate component of approx. 45-60% (Gold, P. and Freedman, S. O., J. Exp Med 121 (1965) 439-462).

CEA, like AFP, belongs to the group of carcinofetal antigens that arc produced during the embryonic and fetal period. The CEA gene family consists of about 17 active genes in two subgroups. The first group contains CEA and the non-specific cross-reacting antigens (NCA); the second group contains the pregnancy-specific glycoproteins (PSG).

CEA is mainly found in the fetal gastrointestinal tract and in fetal serum. It also occurs in slight quantities in intestinal, pancreatic, and hepatic tissue of healthy adults. The formation of CEA is repressed after birth, and accordingly serum CEA values are hardly measurable in healthy adults.

High CEA concentrations are frequently found in cases of colorectal adenocarcinoma (Fateh-Modhadam, A. et al. (eds.), Tumormarker und ihr sinnvoller Einsatz, Juergen Hartmarin Verlag GmbH, Marloffstein-Rathsberg (1993), ISBN-3-926725-07-9). Slight to moderate CEA elevations (rarely >10 ng/mL) occur in 20-50% of benign diseases of the intestine, the pancreas, the liver, and the lungs (e.g., liver cirrhosis, chronic hepatitis, pancreatitis, ulcerative colitis, Crohn's Disease, emphysema (Fateh-Moghadam, A., et al., supra). Smokers also have elevated CEA values.

The main indication for CEA determinations is therapy management and the follow-up of patients with colorectal carcinoma.

CEA determinations are not recommended for cancer-screening in the general population. CEA concentrations within the normal range do not exclude the possible presence of a malignant disease.

The antibodies in the assay manufactured by Roche Diagnostics react with CEA and (as with almost all CEA detection methods), with the meconium antigen with NCA1 is 0.7% (Hammarstrom, S., et al., Cancer Res. 49 (1989) 4852-4858; and Bormer, O. R., Tumor Biol. 12 (1991) 9-15).

CEA has been measured on an ELECSYS analyzer (Roche Diagnostics GmbH) using Roche product number 11731629 according to the manufacturer's instructions.

As used, herein, each of the following terms has the meaning associated with it in this section.

The articles “a” and “an” are used herein to refer to one or to more than one (i.e. to at least one) of the grammatical object of the article. By way of example, “a marker” means one marker or more than one marker.

The term “marker” or “biochemical marker” as used herein refers to a molecule to be used as a target for analyzing patient test samples. Examples of such molecular targets are proteins or polypeptides themselves as well as antibodies present in a sample. Proteins or polypeptides used as a marker in the present invention are contemplated to include any variants of said protein as well as fragments of said protein or said variant, in particular, immunologically detectable fragments. One of skill in the art would recognize that proteins which are released by cells or present in the extracellular matrix which become damaged, e.g., during inflammation could become degraded or cleaved into such fragments. Certain markers are synthesized in an inactive form, which may be subsequently activated by proteolysis. As the skilled artisan will appreciate, proteins or fragments thereof may also be present as part of a complex. Such complex also may be used as a marker in the sense of the present invention. Variants of a marker polypeptide are encoded by the same gene, but differ in their PI or MW, or both (e.g., as a result of alternative mRNA or pre-mRNA processing, e.g., alternative splicing or limited proteolysis) and in addition, or in the alternative, may arise from differential post-translational modification (e.g., glycosylation, acylation, and/or phosphorylation).

The term “assessing colorectal cancer” is used to indicate that the method according to the present invention will (alone or together with other methods or variables, e.g., the criteria set forth by the UICC (see above)), e.g., aid the physician to establish or confirm the absence, or presence of CRC or aid the physician in the prognosis, the monitoring of therapy efficacy (e.g., after surgery, chemotherapy or radiotherapy) and the detection of recurrence (follow-up of patients after therapy).

The term “sample” as used herein refers to a biological sample obtained for the purpose of evaluation in vitro. In the methods of the present invention, the sample or patient sample preferably may comprise any body fluid. Preferred test samples include blood, serum, plasma, urine, saliva, and synovial fluid. Preferred samples are whole blood, serum, plasma or synovial fluid, with plasma or serum being most preferred.

As the skilled artisan will appreciate, any measurement and corresponding assessment is made in vitro. The patient sample is discarded afterwards. The patient sample is solely used for the in vitro diagnostic method of the invention and the material of the patient sample is not transferred hack into the patient's body. Typically, the sample is a liquid sample, e.g., whole blood, serum, or plasma.

The ideal scenario for diagnosis would be a situation wherein a single event or process would cause the respective disease as, e.g., in infectious diseases. In all other cases correct diagnosis can be very difficult, especially when the etiology of the disease is not fully understood as is the case for CRC. As the skilled artisan will appreciate, no biochemical marker, for example in the field of CRC, is diagnostic with 100% specificity and at the same time 100% sensitivity for a given disease. Rather biochemical markers, e.g., are used to assess with a certain likelihood or predictive value the presence or absence of a disease. Therefore in routine clinical diagnosis, generally various clinical symptoms and biological markers are considered together in the diagnosis, treatment and management of the underlying disease.

Biochemical markers can either be determined individually or in a preferred embodiment of the invention they can be measured simultaneously using a chip or a bead based array technology. The concentrations of the biomarkers are then interpreted independently using an individual cut-off for each marker or they are combined for interpretation. Preferably the values, measured for CEA and osteopontin are combined using appropriate mathematical or statistical functions.

The marker combination disclosed in the present invention comprising osteopontin and CEA may improve the assessment of CRC. The marker combination comprising osteopontin and CEA may especially be of advantage in one or more of the following aspects: screening; diagnostic aid; prognosis; monitoring of therapy, and follow-up.

### Screening

CRC is the second most common malignancy of both males and females in developed countries. Because of its high prevalence, its long asymptomatic phase and the presence of premalignant lesions, CRC meets many of the criteria for screening. Clearly, a serum tumour marker which has acceptable sensitivity and specificity would be more suitable for screening than either FOB testing or endoscopy.

As the data given in the Examples section demonstrate neither the marker OPN alone nor the marker CEA alone will suffice to allow for a general screening, e.g., of the at risk population for CRC. For both these markers the sensitivity is not high enough at a specificity level required fro screening purposes. The data established in the present invention indicate, however, that the combination of the markers OPN and CEA will form an integral part of a marker panel appropriate for screening purposes. The present invention therefore relates to the use of OPN and CEA as the core of a CRC marker panel for CRC screening purposes. The present data further indicate that certain combinations of these two markers with one or more other marker will be advantageous in the screening for CRC. Therefore the present invention also relates to the use of a marker panel comprising OPN, CEA, and NSE, or of a marker panel comprising OPN, CEA, and NNMT, e.g., for the purpose of screening for CRC.

### Diagnostic Aid

Preoperative CEA values are of limited diagnostic value. Nonetheless the European Committee on Tumor Markers (ECTM) recommends that CEA should be measured before surgery in order to establish a baseline value and for assessing the prognosis. The marker combination according to the present invention is expected to be superior to the marker CEA alone. It is therefore expected and represents a preferred embodiment according to the present invention that the marker combination comprising OPN and CEA is used as a diagnostic aid. The marker combination may be an especially good diagnostic aid once baseline values before surgery are established.

The present invention thus also relates to the use of the marker combination OPN and CEA for establishing a baseline value before surgery for CRC.

### Prognosis

The gold standard for determining prognosis in patients with CRC is the extend of disease as defined by the Dukes', TNM or other staging systems. If a marker such as CEA is to be used for predicting outcome, it must: provide stronger prognostic information than that offered by existing staging systems, provide information independent of the existing systems or provide prognostic data within specific subgroups defined by existing criteria, e.g., in Dukes' B or node-negative patients.

Recently, an American Joint Committee on Cancer (AJCC) Consensus Conference suggested that CEA should be added to the TNM staging system for colorectal cancer. The CEA level should be designated as follows: CX, CEA cannot be assessed; CO, CEA not elevated (<5 μg/l) or CEA1, CEA elevated (>5 μg/l) (Compton, C, et al., Cancer 88 (2000) 1739-1757).

In a preferred embodiment the marker combination CEA and OPN is used to prognose the course of disease of patients suffering from CRC. In a further preferred embodiment the preoperative levels of OPN and CEA are combined with one or more other marker for CRC and/or the TNM staging system as recommended for CEA by the AJCC and used in the prognosis of disease out-come of patients suffering from CRC.

### Monitoring of Chemotherapy

A number of reports have described the use of CEA in monitoring the treatment of patients with advanced CRC (for review, see Duffy, M. J., Clin. Chem. 47 (2001) 625-630; Fletcher, R. H., Ann: Int. Med. 104 (1986) 66-73; Anonymous, J. Clin. Oncol. 14 (1996) 2843-2877). Most of these investigations were retrospective, non-randomized and contained small numbers of patients. These studies suggested: a) that patients with a decrease in CEA levels while receiving chemotherapy generally had a better outcome than those patients whose CEA levels failed to decrease and (b) for almost all patients, increases in CEA levels were associated with disease progression.

Due to the data shown in the example section, it has to be expected that the marker combination comprising OPN and CEA will be superior to CEA alone if used for monitoring of chemotherapy. The present invention therefore also relates to the use of a marker combination comprising OPN and CEA in the monitoring of CRC patients under chemotherapy.

### Follow-Up

Approximately 50% of patients who undergo surgical resection aimed at cure, later develop recurrent or metastatic disease (Berman. J. M., et. al., Lancet 355 (2000) 395-399). Most of these relapses occur within the first 2-3 years of diagnosis and are usually confined to the liver, lungs or locoregional areas. Since recurrent/metastatic disease is invariably fatal, considerable research has focused on its identification at an early and thus potentially treatable stage. Consequently, many of these patients undergo a postoperative surveillance program which frequently includes regular monitoring with CEA.

Serial monitoring with CEA has been shown to detect recurrent/metastatic disease with a sensitivity of approximately of 80% at a specificity of approximately 70% and provides an average lead-time of 5 months (for review, see Duffy, M. J., et al., supra, and Fletcher, R. H., supra). Furthermore, CEA was the most frequent indicator of recurrence in asymptomatic patients (Pietra, N., et al., Dis. Colon Rectum 41 (1998) 1127-1133; and Graham, R. A., et al., Ann. Surg. 228 (1998) 59-63) and was more cost-effective than radiology for the detection of potentially curable, recurrent disease. As regards sites of recurrence/metastasis, CEA was most sensitive (almost 100%)for the detection of liver metastasis. On the other hand, CEA was less reliable for diagnosing locoregional recurrences, the sensitivity being only approximately 60% (Moertei, C. G., et al., Jama 270 (1993) 943-947).

As a compromise between patient convenience, costs and efficiency of disease detection, the EGTM Panel like the ASCO Panel (Anonymous, J. Clin. Oncol. 14 (1996) 2843-2877) suggests that CEA testing be carried out every 2-3 months for at least 3 years after the initial diagnosis. After 3 years, testing could be carried out less frequently; e.g., every 6 months. No evidence exists, however, to support this frequency of testing.

As the above discussion of the state of the art shows, the follow-up of patients with CRC after surgery is one of the most important fields of use for an appropriate biochemical marker or an appropriate combination of markers. Due to the high sensitivity of the marker combination OPN and CEA in the CRC patients investigated it is expected this marker combination alone or in combination with one or more additional marker will be of great help in the follow-up of CRC patients, especially in CRC patients after surgery. The use of a marker panel comprising OPN and CEA, and optionally one or more other marker of CRC in the follow-up of CRC patients represents a further preferred embodiment of the present invention.

The present invention discloses and therefore in a preferred embodiment relates to the use of the markers OPN and CEA in the diagnostic field of CRC or in the assessment of CRC, respectively.

In yet a further preferred embodiment the present invention relates to the use of a marker panel comprising OPN and CEA in combination with one or more marker molecules for colorectal cancer in the assessment of colorectal cancer from a liquid sample obtained from an individual. In this regard, the expression “one or more” denotes 1 to 20, preferably 1 to 10, preferably 1 to 5; more preferred 3 or 4. OPN and CEA and the one or more other marker form a CRC marker panel.

Thus, a preferred embodiment of the present invention is the use of the marker combination OPN and CEA in colorectal cancer in combination with one or more marker molecules for colorectal cancer in the assessment of colorectal cancer from a liquid sample obtained from an individual. Preferred selected other CRC markers with which the measurement of OPN and CEA may be combined are NSE, ASC, NNMT, CA 19-9, MASP, CYFRA 21-1, FREE and/or CA 72-4. Yet further preferred the marker panel used in the assessment of CRC comprises OPN and CEA and at least one other marker molecule selected from the group consisting of NSE and NNMT.

The preferred one or more other marker(s) that are is/are combined with OPN and CEA or which form part of the CRC marker panel comprising OPN and CEA, respectively, are discussed in more detail below.

### NSE

NSE (neuron-specific enolase), also known as the glycolytic enzyme enolase (2-phospho-D-glycerate hydrolase, EC 4.2.1.11, molecular weight approx. 80 kD) occurs in a variety of dimeric isoforms comprising three immunologically different subunits termed α, β, and γ. The α-subunit of enolase occurs in numerous types of tissue in mammal's, whereas the β-subunits found mainly in the heart and in striated musculature. The enolase isoforms αγ and γγ, which are referred to as neuron-specific enolase (NSE) or γ-enolase, are primarily-detectable in high concentrations in neurons and neuro-endocrine cells as well as in tumors originating from them. (Lamerz R., NSE (Neuronen-spezifische Enolase), γ-Enolase, In: Clinical Laboratory Diagnosis. Thomas, L. (ed.), TH-Books. Frankfurt, 1^{st} English edition (1998), pp. 979-981, 5. deutsche Auflage (1998) pp. 1000-1003).

NSE is described as the marker of first choice in the monitoring of small cell bronchial carcinoma (Lamerz, R., NSE (Neuronen-spezifische Enolase), γ-Enolase, supra), whereas CYFRA 21-1 is superior to NSE for non-small cell bronchial carcinoma (Ebert, W., et al., Eur. J. Clin. Chem. Clin. Biochem. 32 (1994) 189-199).

Elevated NSE concentrations are found in 60-81% of cases of small cell bronchial carcinoma.

For NSE there is no correlation to the site of metastasis or to cerebral metastasis; but there is good correlation to the clinical stage, i.e., the extent of the disease.

In response to chemotherapy there is a temporary rise in the NSE level 24-72 hours after the first therapy cycle as a result of cytolysis of the tumor cells. This is followed within a week or by the end of the first therapy cycle by a rapid fall in the serum values (which were elevated prior to therapy). By contrast, non-responders to therapy display levels which are constantly elevated or fail to fall into the reference range. During remission, 80-96% of the patients have normal values. Rising NSE values are found in cases of relapse. The rise occurs in some cases with a latent period of 1-4 months, is often exponential (with a doubling time of 10-94 days) and correlates with the survival period. NSE is useful as a single prognostic factor and activity marker during the monitoring of therapy and the course of the disease in small cell bronchial carcinoma: diagnostic sensitivity 93%, positive predictive value 92% (Lamerz, R., NSE (Neuronen-spezifische Enolase), γ-Enolase, supra).

In neuroblastoma NSE serum values above 30 ng/ml are found in 62% of the affected children. The medians rise in accordance with the stage of the disease. There is a significant correlation between the magnitude or frequency of pathological NSE values and the stage of disease; there is an inverse correlation with illness-free survival.

68-73% of the patients with seminoma have a clinically significant NSE elevation (Lamerz, R., NSE (Neuronen-spezifische Enolase), γ-Enolase, supra). There is a utilizable correlation with the clinical course of the disease.

NSE has also been measured in other tumors: Non-pulmonary malignant diseases show values above 25 ng/ml in 22% of the cases (carcinomas in all stages). Brain tumors such as glioma, miningioma, neurofibroma, and neurinoma are only occasionally accompanied by elevated serum NSE values. In primary brain tumors or brain metastasis and in malignant melanoma and phaeochromocytoma, elevated NSE-values can occur in the CSF (cerebrospinal fluid). Increased NSE concentrations have been reported for 14% of organ-restricted and 46% of metastasizing renal carcinomas, with a correlation to the grade as an independent prognosis factor.

In benign disease elevated serum NSE concentrations (>12 ng/ml) have been found in patients with benign pulmonary diseases and cerebral diseases. Elevated values, mainly in the liquor, have been found in cerebrovascular meringitis, disseminated encephalitis, spinocerebellar degeneration, cerebral ischemia, cerebral infarction, intracerebral hematoma, subarachnoid hemorrhage, head injuries, inflammatory brain diseases, organic epilepsy, schizophrenia, and Jakob-Creutzfeld disease (Lamerz, R., NSE (Neuronen-spezifische Enolase), γ-Enolase, supra).

NSE may, e.g., be measured on an ELECSYS analyzer using Roche product number 12133113 according to the manufacturer's instructions.

### NNMT

The protein nicotinamide N-methyltransferase (NNMT; Swiss-PROT: P40261) has an apparent molecular weight of 29.6 kDa and an isoelectric point of 5.56.

NNMT catalyzes the N-methylation of nicotinamide and other pyridines. This activity is important for biotransformation of many drugs and xenobiotic compounds. The protein has been reported to be predominantly expressed in liver and is located in the cytoplasm. NNMT has been cloned from cDNA from human liver and contained a 792-nucleotide open reading frame that encoded a 264-amino acid protein with a calculated molecular mass of 29.6 kDa (Aksoy, S., et al., J. Biol. Chem. 269 (1994) 14835-14840). Little is known in the literature about a potential role of the enzyme in human cancer. In one paper, increased hepatic NNMT enzymatic activity was reported as a marker for cancer cachexia in mice (Okamura, A., et al., Jpn. J. Cancer Res. 89 (1998) 649-656). In a recent report, down-regulation of the NNMT gene in response to radiation in radiation sensitive cell lines was demonstrated (Kassem, H., et al., Int. J. Cancer 101 (2002) 454-460):

It has recently been found (WO 2004/057336) that NNMT will be of interest in the assessment of CRC. The immunoassay described in WO 2004/057336 has been used to measure the samples (CRC, healthy controls and non-malignant colon diseases) of the present study.

### CA 19-9

The CA 19-9 (carbohydrate antigen 19-9) values measured are defined by the use of the monoclonal antibody 1116-NS-19-9. The 1116-NS-19-9-reactive determinant in serum is mainly expressed on a mucin-like protein that contains a high number of CA19-9 epitopes (Magnani, J. L., Arch. Biochem. Biophys. 426(2004) 122-131).

3-7% of the population have the Lewis a-negative/b-negative blood group configuration and are unable to express the mucin with the reactive determinant CA 19-9. This must be taken into account when interpreting the findings.

CA19-9 containing mucins are expressed in fetal gastric, intestinal and pancreatic epithelia. Low concentrations can also be found in adult tissue in the liver, lungs, and pancreas (Fateh-Moghadam, A., et al., supra; Herlyn, M., et al., J. Clin. Immunol. 2 (1982) 135-140).

CA 19-9 assay values can assist in the differential diagnosis and monitoring of patients with pancreatic carcinoma (sensitivity 70-87%) (Ritts, R. E., Jr., et al., Int. J. Cancer 33 (1984) 339-345). There is no correlation between tumor mass and the CA 19-9 assay values. However, patients with CA 19-9 serum levels above 10,000 U/mL almost always have distal metastasis.

The determination of CA 19-9 cannot be used for the early detection of pancreatic carcinoma (Steinberg, W. M., et al., Gastroenterology 90 (1986) 343-349).

In hepatobiliary carcinoma the CA 19-9 values provide a sensitivity of 50-75%. The concomitant determination of CA 72-4 and CEA is recommended in case Of gastric carcinoma. In colorectal carcinoma, determination of CEA alone is adequate; only in a limited number of the CEA-negative cases the determination of CA 19-9 can be useful.

As the mucin is excreted exclusively via the liver, even slight cholestasis can lead to clearly elevated CA 19-9 serum levels in some cases. Elevated CA 19-9 values are also found with a number of benign and inflammatory diseases of the gastrointestinal tract and the liver, as well as in cystic fibrosis.

CA 19-9 has been measured on an ELECSYS analyzer using Roche product number 11776193 according to the manufacturer's instructions.

### ASC

The “apoptosis-associated speck-like protein containing a caspase-associated recruitment domain” (ASC), is also known as “target of methylation-induced silencing 1” (TMS1) (Swiss-PROT: Q9ULZ3). ASC has a theoretical molecular weight of 21,627 Da and a theoretical isoelectric point of pH 6.29.

Caspase-associated recruitment domains (CARDs) mediate the interaction between adaptor proteins such as APAF1 (apoptotic protease activating factor 1) and the pro-form of caspases (e.g., CASP 9) participating in apoptosis. ASC is a member of the CARD-containing adaptor protein family.

By immunoscreening a promyelocyte cell line, Masumoto et al. isolated a cDNA encoding ASC. The deduced 195-amino acid protein contains an N-terminal pyrin-like domain (PYD) and an 87-residue C-terminal CARD. Western blot analysis showed expression of a 22-kDa protein and indicated that ASC may have proapoptotic activity by increasing the susceptibility of leukemia cell lines to apoptotic stimuli by anticancer drugs (Masumoto, J., et al., J. Biol. Chem. 274 (1999) 33835-33838).

Methylation-sensitive restriction PCR and methylation-specific PCR (MSP) analyses by Conway et al. indicated that silencing of ASC correlates with hypermethylation of the CpG island surrounding exon1 and that over expression of DNMT1 (DNA cytosine-5-methyltransferase-1) promotes hypermethylation and silencing of ASC. Breast cancer cell lines, but not normal breast tissue, exhibited complete methylation of ASC and expressed no ASC message. Expression of ASC in breast cancer cell lines inhibited growth and reduced the number of surviving colonies. Conway et al. concluded that ASC functions in the promotion of caspase-dependent apoptosis and that over expression of ASC inhibits the growth of breast cancer cells (Conway, K. E., et al., Cancer Research 60 (2000) 6236-6242).

McConnell and Vertino showed that inducible expression of ASC inhibits cellular proliferation and induces DNA fragmentation that can be blocked by caspase inhibitor. Immunofluorescence microscopy demonstrated that induction of apoptosis causes a CARD-dependent shift from diffuse cytoplasmic expression to spherical perinuclear aggregates (McConnell, B. B., and Vertino, P. M., Cancer Research 60 (2000) 6243-6247). Moriani et al. observed methylation of ASC gene not only in breast cancer cells but also in gastric cancer. They suggested a direct role for aberrant methylation of the ASC gene in the progression of breast and gastric cancer involving down-regulation of the proapoptotic. ASC gene (Moriani, R., el al., Anticancer Research 22 (2002) 4163-4168).

Conway et al. examined primary breast, tissues for TMS1 methylation and compared the results to methylation in healthy tissues (Conway K. E., et al., Cancer Research 60 (2000) 6236-6242). Levine et al. found that ASC silencing was not correlated with methylation of specific CpG sites, but rather was associated with dense methylation of ASC CpG island. Breast tumor cell lines containing exclusively methylated ASC copies do not express ASC, while in partially methylated cell lines the levels of ASC expression are directly related to the percentage of methylated ASC alleles present in the cell population (Levine, J. J., et al., Oncogene 22 (2003) 3475-3488).

Virmani et al. examined the methylation status of ASC in lung cancer and breast cancer tissue. They found that aberrant methylation of ASC was present in 46% of breast cancer cell lines and in 32% of breast tumor tissue. Methylation was rare in non-malignant breast tissue (7%) (Virmani, A., et al., Int. J. Cancer 106(2003) 198-204).

Shiohara et al. found out that up-regulation of ASC is closely associated with inflammation and apoptosis in human neutrophils (Shiohara, M., et al., Blood 98 (2001) 229a).

Masumoto et al. observed that high levels of ASC are abundantly expressed in epithelial cells and leucocytes (Masumoto, J., et al., Journal Histochem. Cytochem. 49 (2001) 1269-1275).

An in-house sandwich immunoassay has been developed for measurement of ASC. This assay is performed in a microtiter plate format. Streptavidin-coated microtiter plates are used. A biotinylated polyclonal antibody to ASC is used as a capturing antibody and a digoxigenylated polyclonal antibody to ASC is used as the second specific binding partner in this sandwich assay. The sandwich complex formed is finally visualized by an anti-digoxigenin horseradish peroxidase conjugate and an appropriate peroxidase substrate.

### MASP

The protein MASP (maspin precursor; Swiss-PROT: P36952) is a 42-kDa protein that shares homology with the serpin superfamily of protease inhibitors. Immunostaining studies demonstrate that maspin is found in the extracellular matrix and at the plasma membrane (Zou, Z., et al., Science 263 (1994) 526-529).

The human MASP gene (SERPINB5 of P15) was originally isolated from normal mammary epithelium by subtractive hybridization on the basis of its expression at the mRNA level (Zou et al., supra). Maspin was expressed in normal mammary epithelial, cells but not in most mammary carcinoma cell lines. Zou et al. (supra) showed that its expression reduces the ability of transformed cells to induce tumor formation and metastasis, suggesting that the maspin gene encodes a tumor suppressor.

Bass, R., et al. (J. Biol. Chem. 277 (2002) 46845-46848) characterized eukaryotic maspin and found that it had no protease inhibitory effect against any of the proteolytic systems tested. It did, however, inhibit the migration of both tumor and vascular smooth muscle cells.

Song, S. Y., et al. (Digestive Diseases and Sciences 47 (2002) 1831-1835) studied the expression of maspin in colon cancers by immunohistochemical staining of tissue sections from adenomas, adenocarcinomas and metastatic adenocarcinomas. The immunoreactivity of maspin found by Song et al. (supra) was cytoplasmic, with some nuclear staining. More than 90% of adenoma, 75% of adenocarcinoma and 47% of metastatic carcinoma tissue sections stained positive for maspin. This study had the limitation that no quantitative assay system, such as western blot analysis, was used. The level of expression in comparison to the adjacent normal colon tissue was not assessed.

### FERR

Ferritin (FERR) is a protein containing about 20% iron and is found in the intestines, the liver and the spleen. It is one of the chief forms in which iron is stored in the body. Body iron stores have been reported to increase the risk of colorectal neoplasms. In a study by Scholefield, J. H., et al. (Dis. Colon Rectum 41 (1998) 1029-1032) using samples from 148 patients. (50 patients with proven colorectal cancer, 49 patients without colon disease, and patients with adenomas of the colon) serum ferritin was assayed. There were no significant differences in serum ferritin levels among any of the three groups.

### CYFRA 21-1

An assay for “CYFRA 21-1” specifically measures a soluble fragment of cytokeratin 19 as present in the circulation. The measurement of CYFRA 21-1 is typically based upon two monoclonal antibodies (Bodenmueller, H., et al., Int. J. Biol. Markers 9 (1994) 75-81). In the CYFRA 21-1 assay from Roche Diagnostics, Germany, the two specific monoclonal antibodies (KS 19.1 and BM 19.21) are used and a soluble fragment of cytokeratin 19 having a molecular weight of approx. 30,000 Daltons is measured.

Cytokeratins are structural proteins forming the subunits of epithelial intermediary filaments. Twenty different cytokeratin polypeptides have so far been identified. Due to their specific distribution patterns they are eminently suitable for use as differentiation markers in tumor pathology. Intact cytokeratin polypeptides are poorly soluble, but soluble fragments can be detected in serum (Bodenmueller, H., et al., supra).

CYFRA 21-1 is a well-established marker for Non-Small-Cell Lung Carcinoma (NSCLC). The main indication for CYFRA 21-1 is monitoring the course of non-small cell lung cancer (NSCLC) (Sturgeon, C., Clinical Chemistry 48 (2002) 1151-1159).

In primary diagnosis high CYFRA 21-1 serum levels indicate an advanced tumor stage and a poor prognosis in patients with non-small cell lung cancer (van der Gaast, A., et al. Dr. J. Cancer 69 (1994) 525-528), et al. A normal or only slightly elevated value does not rule out the presence of a tumor.

Successful therapy is documented by a rapid fall in the CYFRA 21-1 serum level into the normal range. A constant CYFRA 21-1 value or a slight or only slow decrease in the CYFRA 21-1 value indicates incomplete removal of a tumor or the presence of multiple tumors with corresponding therapeutic and prognostic consequences. Progression of the disease is often shown earlier by increasing CYFRA 21-1 values than by clinical symptomatology and imaging procedures.

It is accepted that the primary diagnosis of pulmonary carcinoma should be made on the basis of clinical symptomatology, imaging or endoscopic procedures and intraoperative findings. An unclear circular focus in the lung together with CYFRA 21-1 values>30 ng/mL indicates with high probability the existence of primary bronchial carcinoma.

CYFRA 21-1 is also suitable for course-monitoring in myoinvasive cancer of the bladder. Good specificity is shown by CYFRA 21-1 relative to benign lung diseases (pneumonia, sarcoidosis, tuberculosis, chronic bronchitis, bronchial asthma, emphysema).

Slightly elevated values (up to 10 ng/mL) are rarely found in marked benign liver diseases and renal failure. There is no correlation with sex, age or smoking. The values for CYFRA 21-1 are also unaffected by pregnancy.

Recently it has been found that CYFRA also is of use in detecting disease relapse and assessing treatment efficacy in the field of breast cancer (Nakata, B., et al., British J. of Cancer (2004) 1-6).

CYFRA 21-1 preferably is measured on an ELECSYS analyzer using Roche product number 11820966 according to the manufacturer's instructions.

As the skilled artisan will appreciate there are many, ways to use the measurements of two or more markers in order to improve the diagnostic question under investigation. In a quite simple, but nonetheless often effective approach, a positive result is assumed if a sample is positive for at least one of the markers investigated. This may, e.g., the case when diagnosing an infectious disease, like AIDS.

Frequently, however, the combination of markers is evaluated. Preferably the individual values measured for markers of a marker panel are combined and the combined value is correlated to the underlying diagnostic question. In the present invention the combination of the markers OPN and CEA is used in the assessment of CRC.

Marker values may be combined by any appropriate state of the art mathematical method. Well-known mathematical methods for correlating a marker combination to a disease employ methods like, discriminant analysis (DA) (i.e. linear-, quadratic-, regularized-DA), Kernel Methods (i.e. SVM), Nonparametric Methods (i.e. k-Nearest-Neighbor Classifiers), PLS (Partial Least Squares), Tree-Based Methods (i.e. Logic Regression, CART, Random Forest Methods, Boosting/Bagging Methods), Generalized Linear Models (i.e. Logistic Regression), Principal Components based Methods (i.e. SIMCA), Generalized Additive Models, Fuzzy Logic based Methods, Neural Networks and Genetic Algorithms based Methods. The skilled artisan will have no problem in selecting an appropriate method to evaluate a marker combination of the present invention. Preferably the method used in correlating the marker combination of the invention, e.g., to the absence of presence of CRC is selected from DA (i.e. Linear-, Quadratic-, Regularized Discriminant Analysis), Kernel Methods (i.e. SVM), Nonparametric Methods (i.e. k-Nearest-Neighbor Classifiers), PLS (Partial Least Squares), Tree-Based Methods (i.e. Logic Regression, CART, Random Forest Methods, Boosting Methods), or Generalized Linear Models, (i.e. Logistic Regression). Details relating to these statistical methods are found in the following references: Ruczinski. I., et al. J of Computational and Graphical Statistics, 12 (2003) 475-511; Friedman, J. H., J. of the American Statistical Association 84 (1989) 165-175; Hastie, T., et al., The Elements of Statistical Learning; Springer Series in Statistics (2001); Breiman, L., et al., Classification and regression trees, California, Wadsworth (1984); Breiman, L., Random Forests, Machine Learning 45 (2001) 5-32; Pepe, M. S., The Statistical Evaluation of Medical Tests for Classification and Prediction, Oxford Statistical Science Series, 28 (2003); and Duda, R. O., et al., Pattern Classification, Wiley Interscience, 2nd edition (2001).

It is a preferred embodiment of the invention to use an optimized multivariate cut-off for the underlying combination of biological markers and to discriminate state A from state B, e.g., diseased from healthy. In this type of analysis the markers are no longer independent but form a marker panel. It could be established that combining the measurements of OPN and of CEA does significantly improve the diagnostic accuracy for CRC as compared to either marker alone.

Strikingly—at a constant and preset specificity of about 90%—the sensitivity of the marker combination OPN and CEA for diagnosis of CRC has been found to be significantly increased sis compared to each single marker alone.

Accuracy of a diagnostic method is best described by its receiver-operating characteristics (ROC) (see especially Zweig, M. H., and Campbell, G., Clin. Chem. 39 (1993) 561-577). The ROC graph is a plot of all of the sensitivity/specificity pairs resulting from continuously varying the decision thresh-hold over the entire range of data observed.

The clinical performance of a laboratory test depends on its diagnostic accuracy, or the ability to correctly classify subjects into clinically relevant subgroups. Diagnostic accuracy measures the test's ability to correctly distinguish two different conditions of the subjects investigated. Such conditions are for example health and disease or benign versus malignant disease.

In each case, the ROC plot depicts the overlap between the two distributions by plotting the sensitivity versus 1—specificity for the complete range of decision thresholds. On the y-axis is sensitivity, or the true-positive fraction [defined as (number of true-positive test results)/(number of true-positive+number of false-negative test results)]. This has also been referred to as positivity in the presence of a disease or condition. It is calculated solely from the affected subgroup. On the x-axis is the false-positive fraction, or 1-specificity [defined as (number of false-positive results)/(number of true-negative+number of false-positive results)]. It is an index of specificity and is calculated entirely from the unaffected subgroup. Because the true- and false-positive fractions are calculated entirely separately, by using the test, results from two different subgroups, the ROC plot is independent of the prevalence of disease in the sample. Each point on the ROC plot represents a sensitivity/1-specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left corner to the upper right corner. Most plots fall in between these two extremes. (If the ROC plot falls completely below the 45° diagonal, this is easily remedied by reversing the criterion for “positivity” from “greater than” to “less than” or vice versa.) Qualitatively, the closer the plot is to the upper left corner, the higher the overall accuracy of the test.

One convenient goal to quantify the diagnostic accuracy of a laboratory test is to express its performance by a single number. Such an overall parameter, e.g., is the so-called “total error” or alternatively the “area under the curve=AUC”. The most common global measure is the area under the ROC plot. By convention, this area is always ≧0.5 (if it is not, one can reverse the decision rule to make it so). Values range between 1.0 (perfect separation of the test values of the two groups) and 0.5 (no apparent distributional difference between the two groups of test values). The area does not depend only on a particular portion of the plot such as the point closest to the diagonal or the sensitivity at 90% specificity, but on the entire plot. This is a quantitative, descriptive expression of how close die ROC plot is to the perfect one (area=1.0):

The combination of the two markers OPN and CEA significantly improves the diagnostic accuracy for CRC as demonstrated by an increased area under the curve.

Combining measurements of OPN and CEA with other recently discovered markers for CRC, like ASC or NNMT or with known tumor markers like CYFRA 21-1, and NSE, or with other markers of CRC yet to be discovered, leads and will lead, respectively, to further improvements in assessment of CRC.

In a preferred embodiment the present invention relates to a method for improving the diagnostic accuracy for CRC versus healthy controls and patients suffering from non-malignant colon disease by measuring in a sample the concentration of at least OPN and CEA, respectively, mathematically combining the values measured and correlating the concentrations determined to the presence or absence of CRC, the improvement resulting in more patients being correctly classified as suffering from CRC versus healthy controls and patients suffering from non-malignant colon disease as compared to a classification based on a single marker alone.

In yet a further preferred method according to the present invention at least the concentration of the biomarkers OPN, CEA and NSE, respectively, is determined and the marker combination is used in the assessment of CRC.

In yet a further preferred method according to the present invention at least the concentration of the biomarkers OPN, CEA and NNMT, respectively, is determined and the marker combination is used in the assessment of CRC.

The following examples are provided to aid the understanding of the present invention, the true scope of which is set forth in the appended claims. It is understood that modifications can be made in the procedures set forth without departing from the spirit of the invention.

### EXAMPLE 1

### Study Population

The study population is given in Table 1.

TABLE 1
Study population: CRC samples and corresponding UICC classification

| Stage according to UICC | Number of samples |
|---|---|
| UICC 0 | 8 |
| UICC I | 41 |
| UICC II | 53 |
| UICC III | 67 |
| UICC I-III (unclassified, non-IV stages) | 13 |
| UICC IV | 61 |
| without staging | 11 |
| total number of CRC samples | 254 |

The study population comprised serum samples from 254 patients diagnosed with CRC (see Table 1) and 391 control samples. Both these groups were split into a training set and a test set.

The analysis was based on a training set of 128 CRC samples and 195 control samples. Of the controls 16 were from individuals without any gastro-intestinal disease, 50 from individuals with hemorrhoids, 5from patients with other bowel diseases; 63 controls came from individuals with diverticulosis, 61 from healthy blood donors.

The test set consisted of 126 CRC samples and 196 controls. Of the controls 20 were from individuals without any gastro-intestinal disease, 43 from individuals with hemorrhoids, 8 from patients with other bowel diseases; 65 controls came from individuals with diverticulosis, 60 from healthy blood donors.

### EXAMPLE 2

### Assay Procedures Used

The markers CEA, CYFRA 21-1, and NSE have been analyzed with commercially available kits (Roche Diagnostics product numbers 11731629, 11820966, and 12133113, respectively).

The immunoassay described in WO 2004/057336 has been used to measure NNMT in the samples of the present study. In brief, for detection of NNMT in human serum or plasma, a sandwich ELISA was developed. For capture and detection of the antigen, aliquots of an anti-NNMT polyclonal antibody were conjugated with biotin and digoxigenin, respectively.

Streptavidin-coated 96-well microtiter plates were incubated with 100 μl biotinylated anti-NNMT polyclonal antibody for 60 min at 10 μg/ml in 10 mM phosphate, pH 7.4, 1% BSA, 0.9% NaCl and 0.1% TWEEN 20 (ICI Americas Inc.). After incubation, plates were washed three times with 0.9% NaCl, 0.1% TWEEN 20. Wells were then incubated for 2 h with either a serial dilution of the recombinant protein (see Example 2) as standard antigen or with diluted plasma samples from patients. After binding of NNMT, plates were washed three times with 6.9% NaCl, 0.1% TWEEN 20. For specific detection of bound NNMT, wells were incubated with 100 μl of digoxigenylated anti-NNMT polyclonal antibody for 60 min at 10 μg/ml in 10 mM phosphate, pH 7.4, 1% BSA, 0.9% NaCl and 0.1% TWEEN 20. Thereafter, plates were washed three times to remove unbound antibody. In a next step, wells were incubated with 20 mU/ml anti-digoxigenin-POD conjugates (Roche Diagnostics GmbH, Mannheim, Germany, Catalog No. 1633716) for 60 min in 10 mM phosphate, pH 7.4, 1% BSA, 0.9% NaCl and 0.1% TWEEN 20. Plates were subsequently washed three times with the same buffer. For detection of antigen-antibody complexes, wells were incubated with 100 μl ABTS solution (Roche Diagnostics GmbH, Mannheim, Germany, Catalog No. 11685767) and OD was measured after 30-60 min at 405 nm with an ELISA reader.

OPN was measured by an in-house sandwich ELISA. For capture and detection of the antigen, two different antibodies were used. These antibodies were selected to have different non-overlapping epitopes. The epitopes of the two antibodies used are between amino acid 167 and the carboxy terminus of the osteopontin sequence (Kiefer M. C., et al., Nucl. Acids Res. 17 (1989) 3306).

One antibody has been biotinylated and used as a capture antibody. The second antibody has been digoxigenylated. The digoxigenylated antibody was then detected by use of an appropriate anti-DIG secondary antibody.

The assay procedure was essentially as described above for the detection of NNMT but for the OPN-specific antibodies.

### EXAMPLE 3

### Mathematical Evaluation of the Data Generated

The classification algorithms were generated with the Regularized Discriminant Analysis (RDA), which is a generalization of the common Discriminant Analysis, i.e., Quadratic- and Linear Discriminant Analysis (McLachlan, G. J., Discriminant Analysis and Statistical Pattern Recognition, Wiley Series in probability and mathematical statistics, 1992). In the RDA alternatives to the usual maximum likelihood (plug-in) estimates for the covariance matrices are used. These alternatives are characterized by two parameters (λ, γ)the values of which are customized to individual situations by jointly minimizing a sample-based estimate of future misclassification risk (Friedman, J. H., Regularized Discriminant Analysis, J. of the American Statistical Association 84 (1989) 165-175). As an alternative method Support Vector Machines algorithms (Hastie, T., et al., The Elements of Statistical Learning, Springer Series in Statistics, 2001) can be fitted with comparable classification results.

The marker panels were stepwise constructed starting from the best single marker for the classification problem and ending when the increase in the sensitivity at a specificity level of about 90% does not change remarkably any more. In order to gain centralized distributions every single marker was transformed with the natural logarithmic function. 5-Fold cross validation was used.

Table 2 presents the classification results of patients diagnosed with CRC versus controls including non-malignant colon diseases.

TABLE 2
Classification results of patients with CRC versus healthy controls and disease controls

| | | | | Cross validation | | Classification of test set | |
|---|---|---|---|---|---|---|---|
| No. of | Marker or marker | | Cut- | (5-fold/training set) | | correct pos. | correct neg. |
| Markers | panel | Method (RDA) | off | sensitivity | specificity | (sensitivity) | (specificity) |
| 1 | log_CEA | λ = 0.5, γ = 0 | 0.6 | 38.2% | 91.2% | 41.3% | 91.8% |
| 1 | log_OPN_T | λ = 0.5, γ = 0 | −0.4 | 34.1% | 90.9% | 30.2% | 92.3% |
| 2 | log_OPN_T | λ = 1, γ = 0 | 0.3 | 45.6% | 90.2% | 48.4% | 91.3% |
| | log_CEA | | | | | | |
| 3 | log_OPN_T | λ = 1, γ = 0 | 0.2 | 47.2% | 90.8% | 46.8% | 90.8% |
| | log_CEA | | | | | | |
| | log_NNMT | | | | | | |
| 3 | log_OPN_T | λ = 0.75, γ = 0 | 0 | 47.1% | 90.9% | 50% | 92.3% |
| | log CEA | | | | | | |
| | log_NSE | | | | | | |

As determined by RDA, the sensitivity for OPN in the above study population was about 34% whereas for CEA a sensitivity of about 38% was found. The marker panel with OPN and CEA alone was found to exhibit a pronounced increase in sensitivity to about 46%. As can be seen from Table 2, the data established with the training set have been essentially confirmed in the test set of samples.

What is claimed is:

1. A method for assessing colorectal cancer in a patient comprising the steps of
providing a sample from the patient,
measuring in the sample a concentration of osteopontin,
measuring in the sample a concentration of carcinoembryonic antigen, and
correlating the concentrations measured to known concentrations of osteopontin and carcinoembryonic antigen in a patient population as a means of assessing colorectal cancer in the patient.

2. The method of claim 1 further comprising the steps of measuring a concentration of a marker selected from the group consisting of neuron-specific enolase (NSE), apoptosis-associated speck-like protein containing a caspase-associated recruitment domain (ASC), nicotinamide N-methyltransferase (NNMT), carbohydrate antigen 19-9 (CA 19-9), carbohydrate antigen 72-4 (CA 72-4), maspin precursor (MASP), soluble fragment of cytokeratin 19 (CYFRA 21-1), and ferritin (FERR) and correlating the concentration of the marker to a concentration of the marker known to be associated with the presence of colorectal cancer in a patient population.

3. The method of claim 2 wherein the marker is NSE.

4. The method of claim 2 wherein the marker is NNMT.

5. A kit for performing the method of claim 1 comprising the reagents required to specifically measure osteopontin and carcinoembryonic antigen in the sample from the patient.
